(19) United States
(12) Patent Application Publication
(10) Pub. No.: US 2005/0186188 A1
(43) Pub. Date: Aug. 25, 2005

(54) Compositions containing probiotics and polysaccharides and methods of use

(76) Inventor: Peilin Guo, Gardena, CA (US)

Correspondence Address: MCCARTER & ENGLISH LLP;CITYPLACE I, 185 ASYLUM STREET, HARTFORD, CT, 06103, US

(21) Appl. No.: 10/782,482

(22) Filed: Feb. 19, 2004

(51) Int. Cl. A61K 45/00; A61K 31/715

(52) U.S. Cl. 424/93.45; 514/54

(57) ABSTRACT

Compositions and methods are provided for improving GI tract health by reducing the level of pathogenic bacteria present. The compositions comprise probiotic microorganisms, such as a lactic acid producing bacteria or a yeast and mannanoligosaccharide. Optionally, the compositions may further comprise fructooligosaccharide. Methods for administering the composition to improve GI tract health are also provided.

### FIELD OF THE INVENTION

The present invention relates generally to compositions having a combination of beneficial bacteria and/or yeast (probiotics) and mannose containing polysaccharides, and methods of use thereof for promoting gastrointestinal health, immune support, and general well being in humans. The compositions may also include prebiotic substances.

### BACKGROUND OF THE INVENTION

The health and wellbeing of humans can be positively or negatively influenced by the microorganisms, such as bacteria and yeast, inside the gastrointestinal (GI) tract. These microorganisms can influence human health through the production of metabolic by-products and short chain fatty acids, by stimulation of host immune response, or through other mechanisms. The GI tract typically contains beneficial microflora which aid in GI tract function and provide other health benefits. However, pathogenic or putrefactive microorganisms also inhabit and colonize the GI tract. There is a constant dynamic between beneficial and pathogenic flora populations in the GI, and the latter can become dominant under certain conditions, such as stress, illness, and changes in diet or physiologic alterations in the GI tract.

Gastrointestinal microflora can be modified via the introduction of bacteria and polysaccharides that have direct or indirect effects on the GI microflora. The introduction of beneficial microorganisms, or probiotics, can positively affect GI microorganism composition and improve human health. Additionally, the introduction of a yeast cell component, mannanoligosaccharides (MOS), can directly reduce the pathogenic bacterial count in the GI tract and positively affect human health.

There is a general consensus that probiotics have beneficial effects on human health. The use of certain bacteria species for their beneficial effect on health has been known for some time. In particular, lactic acid-producing bacteria, such as the genera Lactobacillus, Bifidobacterium, Lactocoocus, Pediococcus, Streptococcus, Saccharomyces, have been used in preparations to promote GI tract health. It has been shown in many scientific experiments and clinical studies that probiotics can provide the following health advantages: protecting the host against intestinal infection by pathogenic microorganisms, modulating immune response to optimize host defense mechanisms, alleviate symptoms associated with irritable bowel syndrome (IBS) and inflammatory bowel diseases (IBD), normalizing inflammatory response, reducing lactose intolerance, decreasing the risk for tumor and cancer development, modulating serum cholesterol, improving bowel regularity, reducing allergies, and so on.

Probiotics have been used in foods and dietary supplements to promote good GI tract health or alleviate symptoms of GI tract health problems. For example, U.S. Pat. No. 6,241,983 to Paul et al. describes a composition comprising a probiotic and fiber to promote GI tract health. U.S. Pat. No. 6,203,797 to Perry describes a dietary supplement containing a probiotic, fructooligosaccharides (FOS) and other components for treating irritable bowel syndrome.

Probiotics typically improve GI tract health by increasing growth of beneficial bacteria, which reduce pathogens indirectly by mechanisms such as competitive site attachment in the GI lumen, producing acids and reducing the pH, or producing bacteriocins to inhibit pathogens. Among the disadvantages of these prior probiotic formulations is the inability of the formulations to bind pathogens directly and reduce the level of pathogens in the GI tract directly.

Mannanoligosaccharide (MOS) is one of the subgroup of “Oligosaccharides” which include a range of naturally occurring molecules in plants and microorganisms, such as yeast. MOS is a polymer consisting of mannose monosaccharide units and can be bonded with proteins as a complex. The beneficial effects of MOS are expressed in several aspects: 1) MOS blocks the attachment of pathogens to the host enterocytes and causes the pathogens to be excreted from the GI tract, out of the host body, and hence reduces pathogenic infection of the GI tract; 2) MOS stimulates the production of immunoglobulins (bile IgA, plasma IgG) and the phagocytic activity of macrophages and enhances host immune system defense mechanisms; and 3) MOS promotes the growth of lactic acid bacteria such as Lactobacillus.

Recently, overuse of antibiotics has become a serious concern due to the development of pathogenic bacterial resistance to antibiotics. MOS, administered together with probiotics, can result in a reduction in antibiotic use in humans by providing an alternative means to remove pathogenic bacteria from the GI tract.

MOS is especially effective in removing bacterial strains that have “type 1 fimbriae” structure on their cells, such as certain strains of Escherichia coli and Salmonella typhimurium. Bacterial fimbriae interact with the specific carbohydrate residues of surface glycoproteins on animal cells. The binding to epithelial cells and erythrocytes by several enteric pathogens can be inhibited by mannose and its oligosaccharides, MOS. These mannose-sensitive fimbriae, termed as “type 1 fimbriae” are found in many gram-negative bacteria and are the most common means of adhesion of certain strains of Escherichia and Salmonella. Because binding to the epithelial cells of the intestine is the first step in bacterial colonization and subsequent infection, the inhibition of bacterial adhesion to the intestinal epithelial cells reduces infection of the host.

Although inhibition of adhesion of bacteria to epithelial cells can be achieved by D-mannose, relatively high concentrations of mannose are required to control the colonization of pathogenic bacteria. In addition, mannose can be fermented (i.e. utilized as a food source) by pathogens. MOS, however, can be produced with little cost, and is not fermented by the pathogen. Instead, MOS is excreted before being utilized by pathogens and reduces the total count of intestinal pathogen by “dragging” pathogens out of the host intestines.

Prebiotics, especially, FOS (FrucoOligoSaccharides), have been shown to enhance human health in many ways, including stimulation of bifidobacteria growth (in contrast to MOS which promote the growth of lactobacillus), modulating immune response, and reduce constipation. The compositions of the present invention may optionally include FOS to further enhance the effects in maintaining the health of the GI tract.

Among the objectives of the present invention is to provide compositions comprising probiotics (bacteria or yeast) and mannanoligosaccharides that may provide one or more of the following benefits: inhibiting host infection by pathogens, boosting beneficial bacterial growth, reducing bacterial antibiotic resistance, enhancing immune system response and function.

### SUMMARY OF THE INVENTION

The present invention provides compositions comprising lactic acid producing bacteria and MOS which may be used for promoting gastrointestinal health, immune support, and general well being in humans, and methods for administering the composition. In one embodiment, the compositions preferably comprise from about 1 million to 100 billion colony forming units (CFU) of a lactic acid producing bacteria and/or yeast and from about 50 mg to about 10 g of MOS. The composition may further comprise pharmaceutically acceptable formulation or delivery aids, such as diluents, stabilizers, binders, buffers, lubricants, coating agents, preservatives, emulsifiers or suspension agents. The compositions may be used in dietary supplements or in food and beverage products. Optionally, the composition of the present invention may further comprise between about 20 mg and 25 g of fructooligosaccharides (FOS).

The present invention further provides methods for administering the composition of the present invention to promote gastrointestinal health in humans. The compositions of the present invention may be administered to provide a total daily intake of between about 10⁴ to about 10⁴ CFU of lactic acid producing bacteria and/or yeast, and between about 20 mg to about 5 g of MOS. In a preferred embodiment of the method of the present invention, the composition is administered to achieve a total daily intake of between about 10⁶ to 10¹² CFU of lactic acid producing bacteria and/or yeast and between about 50 mg to about 5 g of MOS. In another embodiment of the method of the present invention, the composition additionally includes FOS, which is preferably administered to achieve a daily intake of between 20 mg and 25 g per day.

Among the advantages of the present invention is that the probiotic and the MOS provide different and complementary mechanisms for removing pathogenic bacteria from the GI tract, enhancing immune function, and therefore provide an improved composition and host defense for treating or maintaining the health of the GI tract and general wellbeing. Other advantages of the compositions and methods of the present invention will be apparent to those skilled in the art based on the detailed description of preferred embodiments set forth below.

### DETAILED DESCRIPTION OF THE INVENTION

As noted previously several types of bacterial species, referred to as probiotics, have been used to promote GI tract health. In particular, probiotic bacteria and yeast have been included in animal feeds and dietary supplements. The present invention is addressed to compositions for promoting GI tract health, immune function and overall body defense mechanism, comprising a lactic acid producing bacteria or yeast, or a combination of bacteria and yeast, and mannanoligosaccharide (MOS).

Any appropriate strains of lactic acid producing bacteria may be used in the present compositions, including bacteria of the genera Lactobacillus, Bifidobacterium, Lactoccus, Pediococcus, Streptococcus and Saccharomyces. Preferred strains of probiotic microorganisms for use in the present invention are Lactobacilus acidophilus, L. bulgaricus, L. breve, L. casei, L. helveticu, L. rhamnosus, Bifidobacterium bifidum, B. infantis, B. longum, B. adolencentis, Streptococcus lactis, S. thermphilus, Lactococcus diacetylactis, Pediococcus acidilactici, and Saccharomyces boulardii. The compositions may have only one strain of lactic acid producing bacteria or, if desired, more than one strain of lactic acid producing bacteria may be included in the composition. Alternatively, an appropriate probiotic yeast may be used in combination with the MOS, or a combination of lactic acid producing bacteria and yeast may be used. In the following discussion and in the claims, the term “probiotic microorganism” means a lactic acid producing bacteria or a yeast.

Mannanoligosaccharides (MOS) are a fraction derived from the cell walls of yeast, such as Saccharomyces cerevisiae. MOS is a non-digestible carbohydrate that is non-hydrolizable by enzymes of the GI tract. For purposes of the present invention, the MOS may be provided in any appropriate form, such as a concentrated, purified form, or in a bonded or non-bonded complex with proteins, in a matrix with beta-glucans, proteins, other carbohydrates or any other fractions of original yeast cell. MOS can prevent the adherence of pathogenic bacteria to GI epithelial cells by preventing their binding to epithelial cells' receptor sites and is then excreted from the body along with pathogens. This effect is enhanced by the presence of the lactic acid producing bacteria in the intestine. The probiotics cause the pH in the area of the intestinal wall to be lowered to a level that is unfavorable for pathogenic bacteria to proliferate in the intestine. This favors the further growth of beneficial microflora in the intestine. Accordingly, by administering a probiotic microorganism in combination with MOS, a synergistic effect is achieved to result in improved removal of pathogenic bacteria from the GI tract and enhanced beneficial microflora population in the intestine.

The compositions of the present invention comprise a lactic acid producing bacteria and mannanoligosaccharide, with the option of inclusion of other probiotic microorganisms such as Saccharomyces boulardii and prebiotics such as FOS. The combination of probiotic microorganisms and mannanoligosaccharide provide a combination of benefits for GI tract health and general wellbeing. Moreover, the combination may provide synergistic effects in that MOS causes certain types of pathogenic bacteria to be excreted from the GI tract via the inhibition of the adhesion of pathogenic bacteria to the epithelial cells, which creates more favorable environment for the growth of probiotic bacteria. Further, MOS can directly promote lactobacillus bacterial growth and improve GI microflora balance. Furthermore, probiotic microorganisms and MOS complement each other in promoting immunoglobulin production and immune response.

The compositions of the present invention comprise sufficient amounts of probiotic microorganisms and MOS to obtain the desired benefits to GI tract and immune health. In a preferred embodiment, the compositions of the present invention contain between about 1 million and about 100 billion CFU of probiotic microorganisms and between about 50 mg and about 10 g MOS per dose. The composition may further comprise formulation aids such as diluents, stabilizers, binders, buffers, lubricants, coating agents, preservatives, emulsifiers and suspension agents.

The composition of the present invention can be in dietary supplements, such as for example tablets, capsules, sachets, chewable tablets, softgels or caplets. The compositions of the present invention may also be used in conjunction with nutritional products such as meal replacement, protein powders, any nutritional powder formulas (such as infant formula, follow-on formula, etc.) or in food such as milk, yogurt, cheese, dairy drinks, or in beverages, such as fruit juices, vegetable juice, and other drinks.

In another embodiment of the present invention, the composition further comprises a prebiotic, such as Fructooligosaccharide (FOS). Prebiotics have been shown to enhance growth of bifidobacteria, modulate immune response, improve digestion and reduce constipation. In this embodiment of the present invention, the composition preferably further contains between about 20 mg and about 25 g FOS per dose.

The compositions of the present invention may be administered to humans to enhance GI tract health. The effective amount to be administered to an individual may vary depending upon the size and age of an individual, and whether the composition is administered to maintain health or to restore health. A person skilled in the art can routinely determine an appropriate dosage and usage of the composition.

In a preferred embodiment of the method of the present invention, the composition is administered to achieve a daily intake of between about 10⁴ to about 10¹⁴ CFU of probiotic microorganisms per day, and between about 50 mg and about 10 g MOS per day. In a more preferred embodiment, the composition is administered to achieve a daily intake of between about 10⁶ to 10¹² CFU of probiotic microorganisms, and between about 50 mg to about 5 g of MOS.

In another embodiment of the method of the present invention, the composition administered to the person further comprises FOS. In this embodiment of the method of the present invention, the composition is administered to achieve a daily intake of between about 20 mg to about 25 g per day FOS. In a preferred embodiment, the composition is administered to achieve a daily intake of between about 100 mg and about 10 g per day FOS.

While preferred embodiments of the present invention have been shown and described, various modifications and substitutions may be made without departing from the scope and spirit of the invention. Accordingly, it is to be understood that the present invention has been described by way of example and not by limitation.

1. A composition for improving gastrointestinal tract health comprising:
(a) a probiotic microorganism; and
(b) mannanoligosaccharide

2. The composition of claim 1, wherein the probiotic microorganism is the lactic acid producing bacteria Lactobacilus acidophilus.

3. The composition of claim 1, wherein the probiotic microorganism is selected from the group consisting of Lactobacilus acidophilus, L.bulgaricus, L.breve, L.casei, L.helveticu, L.rhamnosus, Bifidobacterium bifidum, B.infantis, B.longum, B.adolencentis, Streptococcus lactis, S.thermphilus, Lactococcus diacetylactis, Pediococcus acidilactici, and Saccharomyces boulardii.

4. The composition of claim 3, wherein the composition contains at least about 10,000 CFU of probiotic microorganisms.

5. The composition of claim 3, wherein the composition contains between about 1 million to about 100 billion CFU of probiotic microorganisms.

6. The composition of claim 5, wherein the composition contains between about 50 mg to about 10 g mannanoligosaccharide.

7. The composition of claim 1, further comprising fructooligosaccharide.

8. The composition of claim 7, wherein the composition contains between about 20 mg to about 25 g fructooligosaccharide.

9. The composition of claim 6, further comprising at least one formulation aid selected from the group consisting of diluents, stabilizers, binders, buffers, lubricants, coating agents, preservatives, emulsifiers and suspension agents.

10. The composition of claim 8, further comprising at least one formulation aid selected from the group consisting of diluents, stabilizers, binders, buffers, lubricants, coating agents, preservatives, emulsifiers and suspension agents.

11. A method for improving gastrointestinal tract health in humans comprising administering to the human probiotic microorganisms and mannanoligosaccharide effective to provide beneficial effects to GI tract health.

12. The method of claim 11, wherein the probiotic microorganisms and mannanoligosaccharide are administered to achieve a daily intake of between about 10⁴ to about 10¹⁴ CFU of probiotic microorganisms per day, and between about 50 mg and about 10 g MOS per day.

13. The method of claim 11, wherein the probiotic microorganisms and mannanoligosaccharide are administered to achieve a daily intake of between about 10⁶ to 10¹² CFU of probiotic microorganisms, and between about 50 mg to about 2 g of MOS.

14. The method of claim 11, further comprising administering to the human fructooligosaccharide.

15. The method of claim 12, further comprising administering fructooligosaccharide to achieve a daily intake of between about 20 mg to about 25 g.

16. The method of claim 13, further comprising administering fructooligosaccharide to achieve a daily intake of between about 100 mg to about 10 g.
